(19) United States
(12) Patent Application Publication
(10) Pub. No.: US 2022/0234016 A1
(43) Pub. Date: Jul. 28, 2022

(54) MULTIPURPOSE SINGLE STAGE REACTOR FOR INDUSTRIAL C4 DEHYDROGENATION TECHNOLOGY

(71) Applicant: SABIC Global Technologies B.V., Bergen op Zoom (NL)

(72) Inventors: Muhammad H. HAIDER, Riyadh (SA); Waleed K. AL-DAHLOUS, Riyadh (SA); Ahmed S. AL-ZENAIDI, Riyadh (SA)

(73) Assignee: SABIC Global Technologies B.V., Bergen op Zoom (NL)

(21) Appl. No.: 17/611,288

(22) PCT Filed: May 21, 2020
(86) PCT No.: PCT/IB2020/054808

§ 371 Date: Nov. 15, 2021

Related U.S. Application Data
(60) Provisional application No. 62/855,716, filed on May 31, 2019.

(51) Int. Cl. B01J 8/02; C07C 5/373

(52) U.S. Cl. CPC B01J 8/0285; C07C 5/373

(57) ABSTRACT

Reactors and methods for producing olefins from paraffins are disclosed. A hydrocarbon feed stream comprising one or more alkanes is dehydrogenated to produce one or more alkenes in a dehydrogenation compartment of a reactor. The effluent from the dehydrogenation compartment is flowed into a isomerization compartment of the reactor. One or more of the alkenes is isomerized in the isomerization compartment to reduce the number of alkene isomers in a product stream from the isomerization compartment.

### CROSS REFERENCE TO RELATED APPLICATIONS

This application claims the benefit of U.S. Provisional Patent Application No. 62/855,716, filed May 21, 2019, which is expressly incorporated by reference herein in its entirety.

### FIELD OF INVENTION

The present invention generally relates to reactors and methods for producing olefins from paraffins. More specifically, the present invention relates to reactors with integrated dehydrogenation compartment(s) and isomerization compartment(s) and methods of using the reactors to produce olefins from paraffins.

### BACKGROUND OF THE INVENTION

C₄ olefins, such as isobutene, 1-butene, trans-2-butene, and cis-2-butene, are a group of C₄ hydrocarbons that can be used in various chemical production processes. For instance, isobutene is used for MTBE synthesis by etherification with methanol in the presence of an acidic catalyst. 1-butene can be readily used for producing polybutene via polymerization. Furthermore, 1-butene can be used as a co-monomer in the production of polyethylene. 2-butenes (including trans-2-butene and cis-2-butene) can be used for producing propylene via metathesis and producing of gasoline, butadiene, and/or butanone.

Conventionally, C₄ olefins can be produced by separating crude C₄ refinery streams. However, these crude C₄ streams generally contain a large amount of C₄ paraffins, resulting in high energy consumption for processing these C₄ streams and low production efficiency for C₄ olefins. Additionally, further purifying the 1-butene and 2-butenes obtained from these crude C₄ refinery streams also consumes a large amount of energy due to close boiling points of these C₄ olefins, thereby further increasing the overall production cost for producing high purity C₄ olefins from crude C₄ refinery streams. Another method of producing 1-butene includes dimerization of ethylene. However, the feedstock of this method is ethylene, which is in high demand as a feedstock in the processes of producing various high-value polymeric products. Therefore, using high-valued ethylene for the production of 1-butene can be cost prohibitive.

Overall, while the systems and methods for producing C₄ olefins exist, the need for improvements in this field persists in light of at least the aforementioned drawbacks of the conventional systems and methods.

### BRIEF SUMMARY OF THE INVENTION

A solution to at least some of the above mentioned problems associated with methods of producing C₄ olefins has been discovered. The solution resides in a reactor and a method for producing olefins from corresponding paraffins. Notably, the reactor integrates a dehydrogenation compartment with an isomerization compartment in one reactor shell such that 2-butenes (including trans-2-butene and cis-2-butene) produced by n-butane dehydrogenation can be readily isomerized to produce 1-butene. This can be beneficial for at least eliminating the energy consumption and/or capital expenditure needed for separating 2-butenes from the effluent stream of the dehydrogenation. Moreover, the reactor includes a heating section that provides heat for both the dehydrogenation compartment and the isomerization compartment, thereby reducing total energy consumption for both dehydrogenation and isomerization processes compared to utilizing a separated dehydrogenation reactor and isomerization reactor. Therefore, the method of the present invention provides a technical solution to at least some of the problems associated with the conventional methods for producing C₄ olefins.

Embodiments of the invention include a reactor configured to carry out dehydrogenation and isomerization. The reactor comprises a reactor shell. The reactor comprises a dehydrogenation compartment disposed in the reactor shell and adapted to dehydrogenate hydrocarbons. The reactor further comprises an isomerization compartment disposed in the reactor shell and adapted to isomerize hydrocarbons. An outlet of the dehydrogenation compartment is in fluid communication with an inlet of the isomerization compartment such that effluent from the dehydrogenation compartment flows into the isomerization compartment.

Embodiments of the invention include a reactor configured to carry out dehydrogenation and isomerization. The reactor comprises a reactor shell. The reactor further comprises a dehydrogenation compartment disposed in the reactor shell and adapted to dehydrogenate hydrocarbons. The dehydrogenation compartment has a dehydrogenation catalyst disposed in it. The reactor further comprises an isomerization compartment disposed in the reactor shell and adapted to isomerize hydrocarbons. The isomerization compartment has an isomerization catalyst disposed in it. An outlet of the dehydrogenation compartment is in fluid communication with an inlet of the isomerization compartment such that effluent from the dehydrogenation compartment flows into the isomerization compartment, without any separation mechanism and/or additional processing equipment between the outlet of the dehydrogenation compartment and the inlet of the isomerization compartment. The reactor further comprises a heating unit disposed in the reactor shell, adapted to provide heat to the dehydrogenation compartment and/or the isomerization compartment.

Embodiments of the invention include a method of producing olefins. The method comprises providing a reactor. The reactor comprises a reactor shell, a dehydrogenation compartment disposed in the reactor shell, where a dehydrogenation catalyst is disposed in the dehydrogenation compartment, and an isomerization compartment is disposed in the reactor shell, where an isomerization catalyst is disposed in the isomerization compartment. An outlet of the dehydrogenation compartment is in fluid communication with an inlet of the isomerization compartment. The method further comprises flowing a hydrocarbon feed comprising one or more alkanes into the dehydrogenation compartment. The method further comprises dehydrogenating alkanes of the hydrocarbon feed to form a dehydrogenation compartment effluent comprising one or more alkenes. The method further comprises flowing the dehydrogenation compartment effluent to the isomerization compartment. The method further still comprises isomerizing the one or more alkenes of the dehydrogenation compartment effluent.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1A shows a schematic diagram for a front sectional view of a reactor for producing olefins, according to embodiments of the invention;

FIG. 1B shows a schematic diagram for a top sectional view of a reactor for producing olefins, according to embodiments of the invention; and

FIG. 2 shows a schematic flowchart for a method of producing olefins, according to embodiments of the invention.

### DETAILED DESCRIPTION OF THE INVENTION

Currently, C₄ olefins are produced by separating C₄ refinery streams that contain C₄ olefins and a large amount of C₄ paraffins. However, the energy consumption for separating these streams and producing high purity C₄ olefins is generally high due to close boiling points of 1-butene and 2-butenes. Another method of producing C₄ olefins includes dimerization of ethylene. However, the feedstock ethylene in the dimerization process is in high demand for producing various high-value chemicals. Thus, using ethylene to produce C₄ olefins can be cost prohibitive. N-butane dehydrogenation can be used for producing C₄ olefins, but the product stream of this process includes various of C₄ hydrocarbons, which are difficult to separate from each other, resulting in high production cost for C₄ olefins. The present invention provides a solution to at least some of these problems. The solution is premised on a reactor and a method for producing olefins that integrates a dehydrogenation compartment and an isomerization compartment to dehydrogenate paraffin(s) in the dehydrogenation compartment and isomerize, in the isomerization compartment, one or more olefin(s) in the effluent from the dehydrogenation compartment, thereby producing olefin products that are easier to be separated. This can be beneficial for reducing the energy consumption for separating the olefin products, resulting in reduced production cost. Furthermore, the dehydrogenation compartment and the isomerization compartment share a heating section for providing heat to both of the compartments, which can further reduce energy consumption for producing high purity olefins. Moreover, the disclosed reactor avoids using two separate vessels for the dehydrogenation compartment and the isomerization unit, respectively, thereby reducing capital expenditure required for two separate vessels, and piping and heat insulation between the two separate vessels These and other non-limiting aspects of the present invention are discussed in further detail in the following sections.

### A. Reactor for Producing Olefins

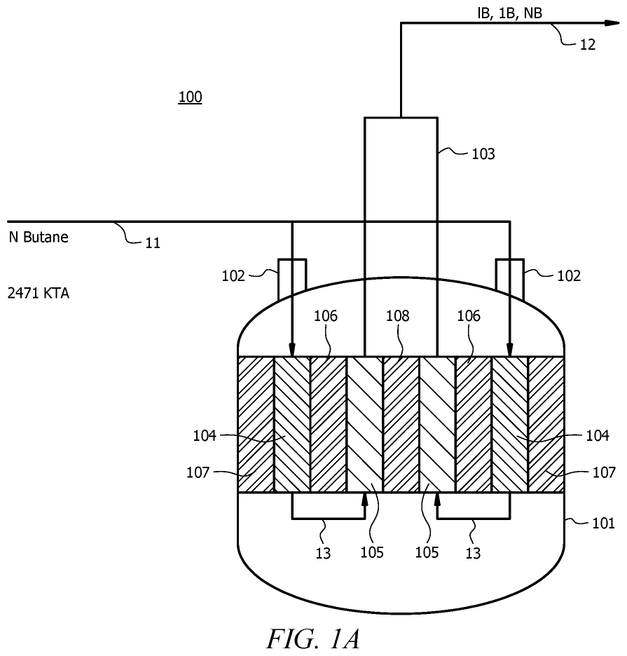

In embodiments of the invention, the reactor for producing olefins can include a reactor shell, a dehydrogenation compartment, and an isomerization compartment. With reference to FIG. 1A, a schematic diagram is shown of reactor 100 that is configured to produce olefins with improved production efficiency and reduced production cost compared to conventional systems and methods. According to embodiments of the invention, reactor 100 is configured to carry out dehydrogenation and isomerization. In embodiments of the invention, reactor 100 is adapted to carry out (i) dehydrogenation of hydrocarbons including one or more alkanes to produce one or more alkenes and (ii) isomerization of the one or more alkene(s).

According to embodiments of the invention, reactor 100 includes reactor shell 101. In embodiments of the invention, reactor shell 101 includes inlet 102 configured to receive feed stream 11 therein. In embodiments of the invention, reactor shell 101 further includes outlet 103 configured to release product stream 12 therefrom. In embodiments of the invention, reactor shell 101 is made of stainless steel. In embodiments of the invention, reactor shell 101 may be in a shape selected from a cylindrical shape, a cubical shape, a rectangular shape, and combinations thereof.

According to embodiments of the invention, reactor 100 includes dehydrogenation compartment 104 disposed in reactor shell 101. In embodiments of the invention, an inlet of dehydrogenation compartment 104 is in fluid communication with inlet 102 of reactor shell 101 such that feed stream 11 flows into dehydrogenation compartment 104. In embodiments of the invention, as shown in FIG. 1B, dehydrogenation compartment 104 may have a top view cross-sectional surface with an annular shape. In embodiments of the invention, dehydrogenation compartment 104 is adapted to dehydrogenate hydrocarbons. In embodiments of the invention, dehydrogenation compartment 104 includes a dehydrogenation catalyst comprising platinum, tin, palladium, gallium, or combinations thereof. In embodiments of the invention, the dehydrogenation catalyst further comprises a supporting material comprising alumina, silica, or combinations thereof. In embodiments of the invention, the dehydrogenation catalyst may comprise a metal to support ratio (wt./wt.) in a range of 0.1:99.09 to 30:70 and all ranges and values there between. In embodiments of the invention, the dehydrogenation catalyst is contained in a fixed catalyst bed.

According to embodiments of the invention, reactor 100 comprises isomerization compartment 105 disposed in reactor shell 101. In embodiments of the invention, an inlet of isomerization compartment 105 is in fluid communication with an outlet of dehydrogenation compartment 104 such that effluent stream 13 flows from dehydrogenation compartment 104 to isomerization compartment 105. In embodiments of the invention, isomerization compartment 105 is configured to isomerize hydrocarbons. According to embodiments of the invention, isomerization compartment 105 is configured to isomerize hydrocarbons including one or more alkenes from effluent stream 13.

According to embodiments of the invention, isomerization compartment 105 comprises an isomerization catalyst including alumina, alpha (α)-alumina, beta (β)-alumina, eta (η)-alumina, or combinations thereof. In embodiments of the invention, the alumina may be η-alumina. In embodiments of the invention, the isomerization catalyst may be contained in a fixed catalyst bed. According to embodiments of the invention, as shown in FIG. 1B, a top view cross-sectional surface of isomerization compartment 105 may have an annular shape. Isomerization compartment 105 and dehydrogenation compartment 104 may have an annular configuration with respect to each other with dehydrogenation compartment 104 as the outer annular compartment. As an alternative configuration to dehydrogenation compartment 104 being the outer annular compartment, in embodiments of the invention, isomerization compartment 105 is the outer annular compartment with respect to dehydrogenation compartment 104. In embodiments of the invention, as shown in FIG. 1B, isomerization compartment 105 and dehydrogenation compartment 104 may be concentric. In embodiments of the invention, an outlet of isomerization compartment 105 is in fluid communication with outlet 103 of reactor shell 101 such that product stream 12 flows from isomerization compartment 105 to exit reactor shell 101.

According to embodiments of the invention, reactor 100 comprises a heating unit disposed in reactor shell 101. The heating unit is configured to provide heat for dehydrogenation compartment 104 and/or isomerization compartment 105. The heating unit may comprise heating sections, including first heating section 106, second heating section 107, and/or third heating section 108, disposed in reactor shell 101. In embodiments of the invention, as shown in FIGS. 1A and 1B, first heating section 106 is disposed between dehydrogenation compartment 104 and isomerization compartment 105. Dehydrogenation compartment 104 may be disposed against an outer surface of first heating section 106. Isomerization compartment may be disposed against an inner surface of first heating section 106. In embodiments of the invention, first heating section 106 may be adapted to concurrently provide heat to both dehydrogenation compartment 104 and isomerization compartment 105.

In embodiments of the invention, second heating section 107 is disposed between dehydrogenation compartment 104 and inner surface of reactor shell 101. Second heating section 107 may be adapted to provide heat to dehydrogenation compartment 104. In embodiments of the invention, third heating section 108 is disposed in space confined by inner wall of isomerization compartment 105. Third heating section 108 may be adapted to provide heat to isomerization compartment 105. According to embodiments of the invention, the heating sections, including first heating section 106, second heating section 107, and third heating section 108, comprise heating coils, heaters, or heating filaments for generating heat.

### B. Method of Producing Olefins

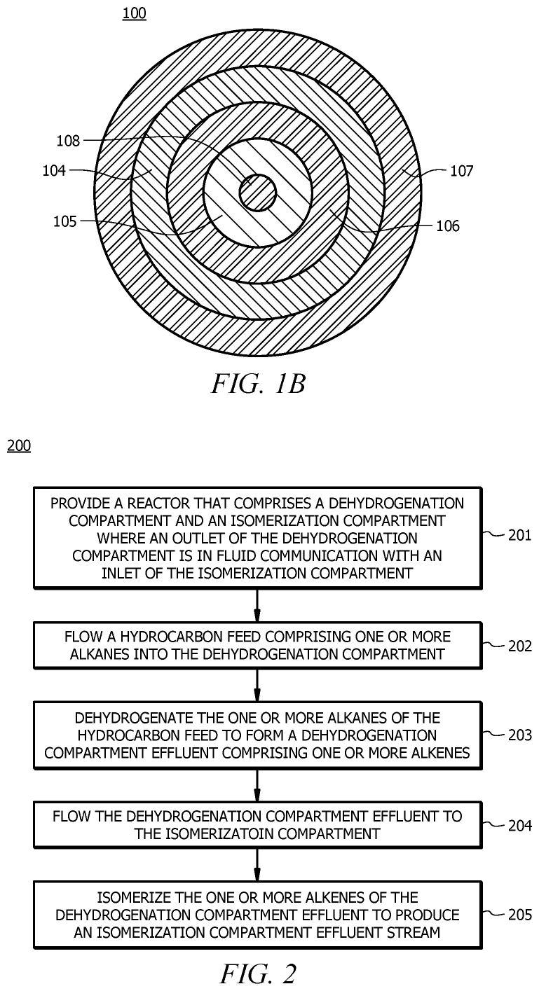

Methods of producing olefins by dehydrogenating alkanes and isomerizing olefins (alkenes) produced by the dehydrogenation have been discovered. Embodiments of the methods are capable of reducing overall production cost for producing olefins compared to conventional methods. As shown in FIG. 2, embodiments of the invention include method 200 for producing olefins. Method 200 may be implemented by reactor 100, as shown in FIGS. 1A and 1B.

According to embodiments of the invention, as shown in block 201, method 200 includes providing reactor 100. In embodiments of the invention, method 200 comprises flowing a hydrocarbon feed (feed stream 11) comprising one or more alkanes into a dehydrogenation compartment 104, as shown in block 202. In embodiments of the invention, the one or more alkanes include n-butane. In embodiments of the invention, feed stream 11 may be at a temperature of 50 to 200° C. and all ranges and values there between including ranges of 50 to 60° C., 60 to 70° C., 70 to 80° C., 80 to 90° C., 90 to 100° C., 100 to 110° C., 110 to 120° C., 120 to 130° C., 130 to 140° C., 140 to 150° C., 150 to 160° C., 160 to 170° C., 170 to 180° C., 180 to 190° C., and 190 to 200° C.

According to embodiments of the invention, as shown in block 203, method 200 includes dehydrogenating the one or more alkanes of the hydrocarbon feed (feed stream 11) to form a dehydrogenation compartment effluent (effluent stream 13) comprising one or more alkenes. In embodiments of the invention, the one or more alkanes in feed stream 11 comprise n-butane and the one or more alkenes comprise butene isomers such as 1-butene, trans-2-butene, cis-2-butene, isobutene, or combinations thereof. In embodiments of the invention, effluent stream 13 may include 20 to 30 wt. % 1-butene, 2 to 5 wt. % isobutene, 25 to 35 wt. % trans-2-butene, 20 to 30 wt. % cis-2-butene, and 30 to 50 wt. % n-butane.

In embodiments of the invention, at block 203, dehydrogenating is performed under reaction conditions comprising a dehydrogenation temperature of 400 to 800° C. and all ranges and values there between including ranges of 400 to 420° C., 420 to 440° C., 440 to 460° C., 460 to 480° C., 480 to 500° C., 500 to 520° C., 520 to 540° C., 540 to 560° C., 560 to 580° C., 580 to 600° C., 600 to 620° C., 620 to 640° C., 640 to 660° C., 660 to 680° C., 680 to 700° C., 700 to 720° C., 720 to 740° C., 740 to 760° C., 760 to 780° C., and 780 to 800° C. The reaction conditions at block 203 may include a dehydrogenation pressure of 0 to 25 bar and all ranges and values there between including ranges of 0 to 2.5 bar, 2.5 to 5.0 bar, 5.0 to 7.5 bar, 7.5 to 10 bar, 10 to 12.5 bar, 12.5 to 15 bar, 15 to 17.5 bar, 17.5 to 20 bar, 20 to 22.5 bar, and 22.5 to 25 bar. Reaction conditions at block 203 may further include a weight hourly space velocity in a range of 1000 to 5000 hr⁻¹ and all ranges and values there between including ranges of 1000 to 1500 hr⁻¹, 1500 to 2000 hr⁻¹, 2000 to 2500 hr⁻¹, 2500 to 3000 hr⁻¹, 3000 to 3500 hr⁻¹, 3500 to 4000 hr⁻¹, 4000 to 4500 hr⁻¹, and 4500 to 5000 hr⁻¹.

According to embodiments of the invention, as shown in block 204, method 200 includes flowing the dehydrogenation compartment effluent (effluent stream 13) to isomerization compartment 105. In embodiments of the invention, effluent stream 13 flows into isomerization compartment 105 through an inlet disposed at the bottom of isomerization compartment 105. According to embodiments of the invention, as shown in block 205, method 200 includes isomerizing the one or more alkenes of the dehydrogenation compartment effluent (effluent stream 13) to produce an isomerization compartment effluent stream (product stream 12). In embodiments of the invention, isomerizing at block 205 includes isomerizing 2-butenes, including trans-2-butene and cis-2-butene to produce 1-butene. In embodiments of the invention, the isomerization compartment effluent (product stream 12) comprises less than 5 wt. % 2-butenes. In embodiments of the invention, the isomerization compartment effluent (product stream 12) comprises substantially no 2-butenes.

In embodiments of the invention, the isomerization compartment effluent (product stream 12) comprises 80 to 90 wt. % 1-butene and all ranges and values there between including ranges of 80 to 81 wt. %, 81 to 82 wt. %, 82 to 83 wt. %, 83 to 84 wt. %, 84 to 85 wt. %, 85 to 86 wt. %, 86 to 87 wt. %, 87 to 88 wt. %, 88 to 89 wt. %, and 89 to 90 wt. %. The isomerization compartment effluent (product stream 12) may further comprise 1 to 5 wt. % isobutene and 30 to 50 wt. % n-butane. In embodiments of the invention, isomerizing at block 205 is performed under reaction conditions including a isomerization temperature in a range of 400 to 800° C. and all ranges and values there between including ranges of 400 to 420° C., 420 to 440° C., 440 to 460° C., 460 to 480° C., 480 to 500° C., 500 to 520° C., 520 to 540° C., 540 to 560° C., 560 to 580° C., 580 to 600° C., 600 to 620° C., 620 to 640° C., 640 to 660° C., 660 to 680° C., 680 to 700° C., 700 to 720° C., 720 to 740° C., 740 to 760° C., 760 to 780° C., and 780 to 800° C. In embodiments of the invention, reaction conditions at block 205 may further include isomerization pressure of 0 to 25 bar and all ranges and values there between including ranges of 0 to 2.5 bar, 2.5 to 5.0 bar, 5.0 to 7.5 bar, 7.5 to 10 bar, 10 to 12.5 bar, 12.5 to 15 bar, 15 to 17.5 bar, 17.5 to 20 bar, 20 to 22.5 bar, and 22.5 to 25 bar. The reaction conditions at block 205 may further include a weight hourly space velocity in a range of 1000 to 5000 hr⁻¹ and all ranges and values there between including ranges of 1000 to 1500 hr⁻¹, 1500 to 2000 hr⁻¹, 2000 to 2500 hr⁻¹, 2500 to 3000 hr⁻¹, 3000 to 3500 hr⁻¹, 3500 to 4000 hr⁻¹, 4000 to 4500 hr⁻¹, and 4500 to 5000 hr⁻¹.

Although embodiments of the present invention have been described with reference to blocks of FIG. 2, it should be appreciated that operation of the present invention is not limited to the particular blocks and/or the particular order of the blocks illustrated in FIG. 2. Accordingly, embodiments of the invention may provide functionality as described herein using various blocks in a sequence different than that of FIG. 2.

In the context of the present invention, at least the following 20 embodiments are described. Embodiment 1 is a reactor configured to carry out dehydrogenation and isomerization. The reactor includes a reactor shell, a dehydrogenation compartment disposed in the reactor shell and adapted to dehydrogenate hydrocarbons, and an isomerization compartment disposed in the reactor shell and adapted to isomerize hydrocarbons, wherein an outlet of the dehydrogenation compartment is in fluid communication with an inlet of the isomerization compartment such that effluent from the dehydrogenation compartment flows into the isomerization compartment. Embodiment 2 is the reactor of embodiment 1, wherein the dehydrogenation compartment has a dehydrogenation catalyst disposed in it. Embodiment 3 is the reactor of embodiment 2, wherein the dehydrogenation catalyst is selected from the group consisting of platinum/tin, palladium, gallium, and combinations thereof. Embodiment 4 is the reactor of any of embodiments 1 to 3, wherein the isomerization compartment has an isomerization catalyst disposed in it. Embodiment 5 is the reactor of embodiment 4, wherein the isomerization catalyst is selected from the group consisting of η-alumina, α-alumina, β-alumina, or combinations thereof. Embodiment 6 is the reactor of any of embodiments 1 to 5, wherein the reactor does not include any separation equipment between the outlet of the dehydrogenation compartment and an inlet of isomerization compartment. Embodiment 7 is the reactor of any of embodiments 1 to 6, wherein the dehydrogenation compartment and the isomerization compartment have an annular configuration with respect to each other. Embodiment 8 is the reactor of any of embodiments 1 to 7, wherein the reactor includes a first heating section providing heat to the dehydrogenation compartment and the isomerization compartment concurrently. Embodiment 9 is the reactor of embodiment 8, wherein the first heating section is disposed between the dehydrogenation compartment and the isomerization compartment. Embodiment 10 is the reactor of embodiment 8, wherein the dehydrogenation compartment is disposed against an outer surface of the first heating section and the isomerization compartment is disposed against an inner surface of the first heating section. Embodiment 11 is the reactor of any of embodiments 8 to 10, wherein the reactor further includes a second heating section disposed between the reactor shell and the dehydrogenation compartment, adapted to provide heat to the dehydrogenation compartment. Embodiment 12 is the reactor of any of embodiments 8 to 11, wherein the first heating section and/or the second heating section contain heaters, heating coils, heating filaments, or combinations thereof.

Embodiment 13 is a method of producing olefins. The method includes providing a reactor that contains a reactor shell, a dehydrogenation compartment disposed in the reactor shell, wherein a dehydrogenation catalyst is disposed in the dehydrogenation compartment, and an isomerization compartment disposed in the reactor shell, wherein an isomerization catalyst is disposed in the isomerization compartment, and wherein an outlet of the dehydrogenation compartment is in fluid communication with an inlet of the isomerization compartment. The method further includes flowing a hydrocarbon feed containing one or more alkanes into the dehydrogenation compartment, and dehydrogenating the one or more alkanes of the hydrocarbon feed to form a dehydrogenation compartment effluent containing one or more alkenes. The method also includes flowing the dehydrogenation compartment effluent to the isomerization compartment, and isomerizing alkenes of the dehydrogenation compartment effluent to produce an isomerization compartment effluent. Embodiment 14 is the method of embodiment 13, wherein the one or more alkanes in the hydrocarbon feed contains n-butane. Embodiment 15 is the method of either of embodiments 13 or 14, wherein the dehydrogenating of the one or more alkanes of the hydrocarbon feed produces butene isomers including 1-butene, trans-2-butene, cis-2-butene, isobutene, or combinations thereof. Embodiment 16 is the method of embodiment 15, wherein the isomerizing step includes isomerizing one or more of the butene isomers to produce 1-butene. Embodiment 17 is the method of embodiment 16, wherein the isomerization compartment effluent contains less than 50 to 60 wt. % trans-2-butene and cis-2-butene, collectively. Embodiment 18 is the method of either of embodiments 16 or 17, wherein the isomerization compartment effluent contains substantially no trans-2-butene and cis-2-butene. Embodiment 19 is the method of any of embodiments 13 to 18, wherein the dehydrogenating is performed under reaction conditions including a dehydrogenation temperature of 400 to 800° C. and a dehydrogenation pressure of 0 to 25 bar. Embodiment 20 is the method of any of embodiments 13 to 19, wherein the isomerizing is performed under reaction conditions including an isomerization temperature of 400 to 800° C. and an isomerization pressure of 0 to 25 bar.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A reactor configured to carry out dehydrogenation and isomerization, the reactor comprising:
a reactor shell; a dehydrogenation compartment disposed in the reactor shell and adapted to dehydrogenate hydrocarbons; and
an isomerization compartment disposed in the reactor shell and adapted to isomerize hydrocarbons, wherein an outlet of the dehydrogenation compartment is in fluid communication with an inlet of the isomerization compartment such that effluent from the dehydrogenation compartment flows into the isomerization compartment.

2. The reactor of claim 1, wherein the dehydrogenation compartment has a dehydrogenation catalyst disposed in it.

3. The reactor of claim 2, wherein the dehydrogenation catalyst is selected from the group consisting of platinum/tin, palladium, gallium, and combinations thereof.

4. The reactor of claim 1, wherein the isomerization compartment has an isomerization catalyst disposed in it.

5. The reactor of claim 4, wherein the isomerization catalyst is selected from the group consisting of η-alumina, α-alumina, β-alumina, or combinations thereof.

6. The reactor of claim 1, wherein the reactor does not include any separation equipment between the outlet of the dehydrogenation compartment and an inlet of isomerization compartment.

7. The reactor of claim 1, wherein the dehydrogenation compartment and the isomerization compartment have an annular configuration with respect to each other.

8. The reactor of claim 1, wherein the reactor comprises a first heating section providing heat to the dehydrogenation compartment and the isomerization compartment concurrently.

9. The reactor of claim 8, wherein the first heating section is disposed between the dehydrogenation compartment and the isomerization compartment.

10. The reactor of claim 8, wherein the dehydrogenation compartment is disposed against an outer surface of the first heating section and the isomerization compartment is disposed against an inner surface of the first heating section.

11. The reactor of claim 8, wherein the reactor further comprises a second heating section disposed between the reactor shell and the dehydrogenation compartment, adapted to provide heat to the dehydrogenation compartment.

12. The reactor of claim 8, wherein the first heating section and/or the second heating section comprise heaters, heating coils, heating filaments, or combinations thereof.

13. A method of producing olefins, the method comprising:
providing a reactor that comprises:
a reactor shell;
a dehydrogenation compartment disposed in the reactor shell, wherein a dehydrogenation catalyst is disposed in the dehydrogenation compartment;
an isomerization compartment disposed in the reactor shell, wherein an isomerization catalyst is disposed in the isomerization compartment, wherein an outlet of the dehydrogenation compartment is in fluid communication with an inlet of the isomerization compartment;
flowing a hydrocarbon feed comprising one or more alkanes into the dehydrogenation compartment;
dehydrogenating the one or more alkanes of the hydrocarbon feed to form a dehydrogenation compartment effluent comprising one or more alkenes;
flowing the dehydrogenation compartment effluent to the isomerization compartment; and
isomerizing alkenes of the dehydrogenation compartment effluent to produce an isomerization compartment effluent.

14. The method of claim 13, wherein the one or more alkanes in the hydrocarbon feed comprises n-butane.

15. The method of claim 13, wherein the dehydrogenating of the one or more alkanes of the hydrocarbon feed produces butene isomers including 1-butene, trans-2-butene, cis-2-butene, isobutene, or combinations thereof.

16. The method of claim 15, wherein the isomerizing step comprises isomerizing one or more of the butene isomers to produce 1-butene.

17. The method of claim 16, wherein the isomerization compartment effluent comprises less than 50 to 60 wt. % trans-2-butene and cis-2-butene, collectively.

18. The method of claim 16, wherein the isomerization compartment effluent comprises substantially no trans-2-butene and cis-2-butene.

19. The method of claim 13, wherein the dehydrogenating is performed under reaction conditions including a dehydrogenation temperature of 400 to 800° C. and a dehydrogenation pressure of 0 to 25 bar.

20. The method of claim 13, wherein the isomerizing is performed under reaction conditions including an isomerization temperature of 400 to 800° C. and an isomerization pressure of 0 to 25 bar.
